(19) United States
(12) Patent Application Publication
(10) Pub. No.: US 2015/0134044 A1
(43) Pub. Date: May 14, 2015

(54) STENT FOR ELECTROTHERMAL TREATMENT

(71) Applicant: BCM Co., Ltd., Koyang-si (KR)

(72) Inventors: Joon-sang KIM, Incheon-Si (KR); Guido COSTAMAGNA, Roma (IT); Ivo BOKOSKI, Latina (IT)

(21) Appl. No.: 14/530,824

(22) Filed: Nov. 2, 2014

(30) Foreign Application Priority Data
Nov. 8, 2013 (KR) 10-2013-0135446
Sep. 26, 2014 (KR) 10-2014-0129432

(51) Int. Cl. A61B 18/14; A61B 18/12; A61F 2/90

(52) U.S. Cl. CPC A61B 18/14; A61F 2/90; A61B 18/1206; A61B 2018/00178
USPC 623/1.16; 606/40

(57) ABSTRACT

A stent comprises at least two or more separated bodies formed by separating in a longitudinal direction a hollow cylindrical body formed by weaving superelastic shape memory alloy wires, the separated bodies connected with each other through an insulator formed of a flexible material, wherein the separated bodies respectively include power connection lines formed by extending the wires from rear sides thereof, and wherein when an electricity generator is connected to the power connection lines of the separated bodies, an electric current flows between the separated bodies to generate electric heat by which the lesion tissue is cauterized.

### CROSS-REFERENCE TO RELATED APPLICATIONS

This patent application claims priority under 35 U.S.C. §119 to Korean Patent Application No. 10-2014-0129432, filed on Sep. 26, 2014, and Korean Patent Application No. 10-2013-0135446, filed on Nov. 8, 2013, in the Korean Intellectual Property Office, the disclosures of which are incorporated by reference herein in their entireties.

### TECHNICAL FIELD

Exemplary embodiments of the present invention relate to a stent for electrothermal treatment that may be inserted into a lesion portion stenosed or obstructed in a lumen of the body to expand the lesion portion and that may apply electric heat to the lesion portion to cauterize the lesion tissue.

### DISCUSSION OF RELATED ART

A lumen may be generally stenosed or obstructed by a disease of the body, and examples thereof include a blockage in the stomach entrance due to fundic cancer, esophageal atresia due to esophageal cancer, vessel stricture due to arteriosclerosis, bile duct stricture, etc.

Such stenosis or blockage of a lumen in the body may cause various complication disease as well as a hypofunction of the lumen.

To address such problems, stents have been used.

A stent, as shown in FIG. 1, includes a hollow cylindrical body 3 formed by weaving superelastic shape memory alloy wires 2, and the stent may thus be extended or contracted in inner or outer directions or in a longitudinal direction. Upon a procedure, the stent may be inserted into the lesion portion where the lumen in the body is stenosed or obstructed to expand the passage of the stenosed or obstructed lesion portion.

However, such conventional stents may merely expand the passage of the stenosed or obstructed lesion portion but cannot provide fundamental treatment on the lesion portion. In other words, in case the stenosed or obstructed lesion portion of the lumen in the body is subjected to progressive disease such as cancer or malignant tumor, the cancer or tumor cell may grow to the inside of inserted stent so that the lumen may be stenosed or obstructed back.

Korean Patent No. 10-0459916 discloses a stent for thermal treatment to address such issues of the conventional stents, wherein a high frequency generator is connected to the stent inserted in the lesion portion where the lumen in the body is stenosed or obstructed to expand the passage of the lesion portion by the stent while applying high frequency heat to the lesion portion to cauterize the lesion portion to thereby lead to necrosis.

However, the invention disclosed in the above-mentioned patent document is configured so that a pad is brought in contact with the patient's body where the stent body is inserted to allow current to flow between the stent body and the pad, thus causing current to flow from the inside of the patient's body to the outside.

In other words, since electric current flows through the organs and skins in the patient's body, the internal organs other than the lesion portion may be negatively affected, and the skin outside the body, on which the pad is attached, may be burned.

### SUMMARY

An embodiment of the present invention is to provide a stent for electrothermal treatment, which may prevent a flow of electric current from the inside of the body to the outside and which may be inserted into a stenosed or obstructed lesion portion in a lumen of the body to expand the lesion portion and to apply electric heat to the lesion portion to cauterize the lesion portion.

According to a first embodiment of the present invention, the present invention is characterized by a stent for electrothermal treatment, the stent inserted into a stenosed or obstructed lesion portion that occurs in a lumen of a body to expand the lesion portion and applying electric heat to the entry point to cauterize a lesion portion, the stent comprising at least two or more separated bodies formed by separating in a longitudinal direction a hollow cylindrical body formed by weaving superelastic shape memory alloy wires, the separated bodies connected with each other through an insulator formed of a flexible material, wherein the separated bodies respectively include power connection lines formed by extending the wires from rear sides thereof, and wherein when an electricity generator is connected to the power connection lines of the separated bodies, an electric current flows between the separated bodies to generate electric heat by which the lesion tissue is cauterized.

The present invention is characterized in that the power connection lines each are formed in a ring shape.

The present invention is characterized in that the power connection lines of the separated bodies each are configured to be inserted and guided in a tube that may be inserted into the body while preventing a contact to skin and that is formed of an insulation polymer.

The present invention is characterized in that the insulator is formed of polytetrafluoroethylene (PTFE) in a tube shape.

Further, according to a second embodiment of the present invention, the present invention is characterized by a stent for electrothermal treatment, the stent comprising a first hollow cylindrical body formed long in a longitudinal direction by weaving or crossing superelastic shape memory alloy wires in a mesh pattern, an end of the first cylindrical body having an expanded tube part larger in diameter than the first cylindrical body to be stuck in a lumen of a body, wherein an insulation layer 60 is formed by coating the first separated bodies with an insulation material, wherein second hollow cylindrical bodies shorter than the first cylindrical body are formed at both sides, respectively, of an outer circumferential surface of the insulation layer, the second cylindrical bodies connected and fixed to the first cylindrical body, wherein power connection lines formed by extending outwards the wires from first ends of the pair of second cylindrical bodies are connected with power lines, respectively, of an electricity generator, and wherein after placing the first cylindrical body on a lesion portion with the expanded tube part stuck in the lumen of the body to thereby expand the narrowed lumen of the body, the power lines of the electricity generator are connected to the power connection lines of the second cylindrical bodies to allow an electric current to flow between the pair of second cylindrical bodies to generate electric heat by which a lesion tissue is cauterized.

According to the first embodiment, the present invention provides the effect that electric current flows only through each separated body without flowing from the inside of the body to the outside thereof.

Further, the present invention provides the effect that the power connection lines extended from the separated bodies are covered by the insulation polymer tube, thus preventing cauterization of unnecessary portions that may occur due to contact with the lumen in the body.

Further, electric current may be prevented from flowing from the power connection line of a separated body to the other separated body, thus providing for adjustment to prevent electric heat from being unnecessarily generated.

Further, the present invention provides the effects that the power connection lines of the separated bodies each are formed in a ring shape to thus allow for power connection despite occurrence of a partial short circuit and that the insulator connecting the separated bodies to each other is formed of an artificial vessel material (polytetrafluoroethylene; PTFE) in a tube shape to thus allow for an effective insertion procedure even on a curvy lumen.

Meanwhile, according to the second embodiment, the present invention provides the effect that the expanded tube part is stuck in the lesion portion at a position of the lumen in the body to prevent it from being slid on the stenosed or obstructed lesion portion by an external force that may occur outside or by body swinging while stably cauterizing the lesion tissue at the same time.

Further, a dual-structure of the first cylindrical body and a second cylindrical body allows for cauterization of the lesion tissue, with the lumen, which has been narrowed due to stenosis or obstruction in the body, expanded more easily.

### BRIEF DESCRIPTION OF DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a front view illustrating an example stent according to a related art;

FIG. 2 is a front view illustrating a stent according to a first embodiment of the present invention;

FIGS. 3A, 3B, and 3C are views illustrating portions “A,” “B,” and “C,” respectively, of the stent shown in FIG. 2;

FIG. 4 is a front view illustrating a stent according to another first embodiment of the present invention;

FIG. 5 is a front view illustrating a stent according to still another first embodiment of the present invention:

FIG. 6 is a front view illustrating an example of using the stent shown in FIG. 2;

FIG. 7 is a front view illustrating an example of using the stent shown in FIG. 4;

FIG. 8 is a front view illustrating an example of using the stent shown in FIG. 5;

FIG. 9 is a front view illustrating a stent according to a second embodiment of the present invention;

FIG. 10 is a front view illustrating a stent according to another second embodiment of the present invention:

FIG. 11 is a front view illustrating an example of using the stent shown in FIG. 9: and

FIG. 12 is a front view illustrating an example of using the stent shown in FIG. 10.

### DETAILED DESCRIPTION OF EMBODIMENTS

Hereinafter, exemplary embodiments of the present invention will be described in detail with reference to the accompanying drawings. The present invention, however, may be modified in various different ways, and should not be construed as limited to the embodiments set forth herein. As used herein, the singular forms “a,” “an,” and “the” are intended to include the plural forms as well, unless the context clearly indicates otherwise. It will be understood that when an element or layer is referred to as being “on,” “connected to,” “coupled to,” or “adjacent to” another element or layer, it can be directly on, connected, coupled, or adjacent to the other element or layer, or intervening elements or layers may be present.

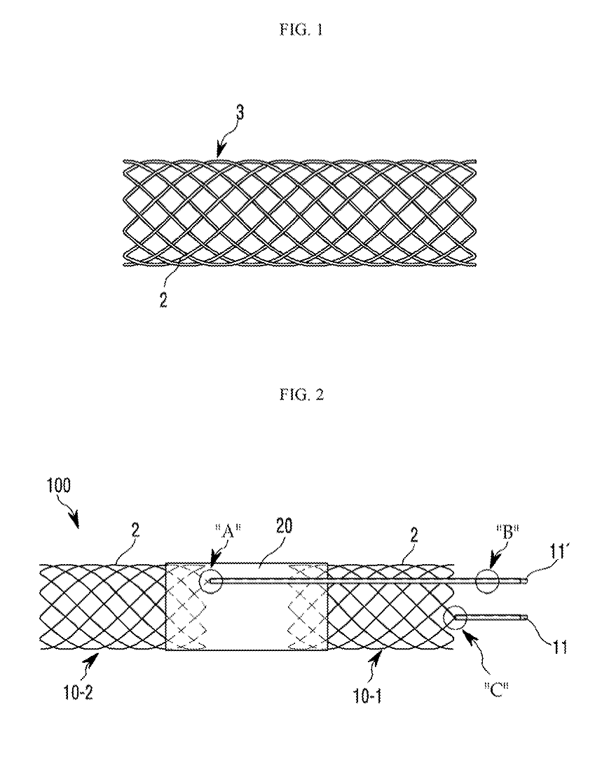

According to a first embodiment of the present invention, a stent 100 for electrothermal treatment may be inserted into a lesion portion 200 stenosed or obstructed in a lumen of the body to expand the lesion portion and to apply electric heat to the lesion portion to cauterize the lesion portion as shown in FIGS. 2 to 8.

A hollow cylindrical body formed by weaving superelastic shape memory alloy wires 2 may be separated into two or more separated bodies in a longitudinal direction, and the separated bodies may be coupled with each other by an insulator 20 formed of a flexible material.

Each separated body may have a power connection line extended from a rear side of the wire 2. If an electricity generator 30 may be connected to the power connection line of each separated body, an electric current may flow between the separated bodies to generate electric heat that may then cauterize the lesion tissue.

For example, as shown in FIG. 2, according to the first embodiment of the present invention, the stent 100 for electrothermal treatment may have two separated bodies, i.e., first and second separated bodies 10-1 and 10-2, and power connection lines 11 and 11′ extended from rear sides of the wires 2 of the first and second separated bodies 10-1 and 10-2. If the electricity generator 30 is connected to the power connection lines 11 and 11′ of the first and second separated bodies 10-1 and 10-2, an electric current flows between the first and second separated bodies 10-1 and 10-2 to generate electric heat that cauterizes the lesion tissue.

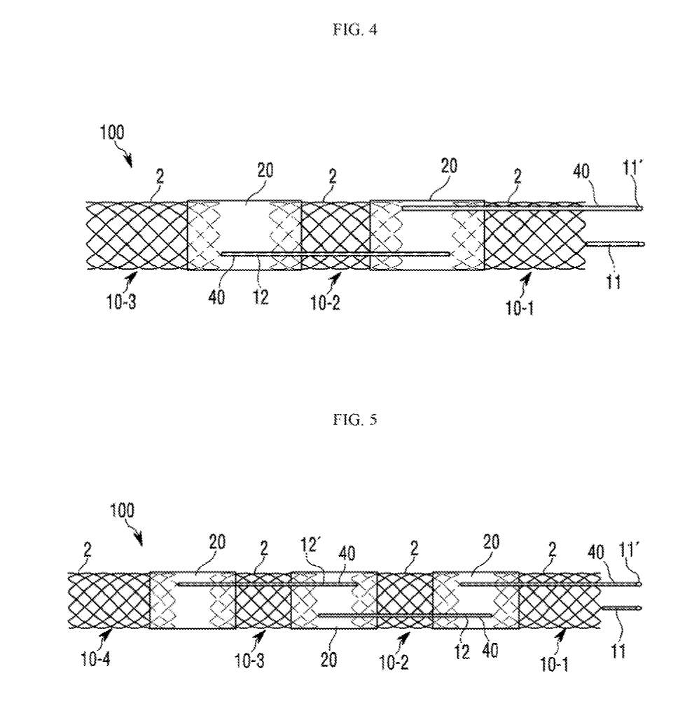

Further, as shown in FIG. 4, according to another first embodiment of the present invention, a stent 100 for electrothermal treatment may include three separated bodies, i.e., first, second, and third separated bodies 10-1, 10-2, and 10-3, power connection lines 11 and 11′ extended from rear sides of the wires 2 of the first and second separated bodies 10-1 and 10-2, respectively, and a power connection line 12 extended from a rear side of the wire 2 of the third separated body 10-3. The power connection line 12 is connected to a front side of the first separated body 10-1. If an electricity generator 30 is connected to the power connection lines 11 and 11′ of the first and second separated bodies 10-1 and 10-2, an electric current flows between the first, second, and third separated bodies 10-1, 10-2, and 10-3 to generate electric heat that cauterizes the lesion tissue.

Further, as shown in FIG. 5, according to still another embodiment of the present invention, a stent 100 for electrothermal treatment may include four separated bodies, i.e., first, second, third, and fourth separated bodies 10-1, 10-2, 10-3, and 10-4, power connection lines 11 and 11′ extended from rear sides of the wires 2 of the first and second separated bodies 10-1 and 10-2, respectively, a power connection line 12 that is extended from a rear side of the wire 2 of the third separated body 10-3 and that is connected to a front side of the first separated body 10-1, and a power connection line 12′ that is extended from a rear side of the wire 2 of the fourth separated body 10-4 and that is connected to a front side of the second separated body 10-2. If an electricity generator 30 is connected to the power connection lines 11 and 11′ of the first and second separated bodies 10-2 and 10-2, an electric current flows between the first, second, third, and fourth separated bodies 10-1, 10-2, 10-3, and 10-4 to generate electric heat that cauterizes the lesion tissue.

Further, according to the present invention, the power connection lines 12 and 12′ of the third and fourth separated bodies 10-3 and 10-4 may be further extended rearwards and selectively connected to the electricity generator 30 separately from the power connection lines 11 and 11′ in order to perform cauterization. However, it may be difficult to connect each of multiple separated bodies to the electricity generator 30 in the lumen of the body, and thus, it may be preferable to make such connections largely at two positions.

Although each separated body is connected with another non-adjacent separated body, such connection may be made between separated bodies adjacent to each other so that electric heat may be generated depending on the circumstance of the lesion portion 200.

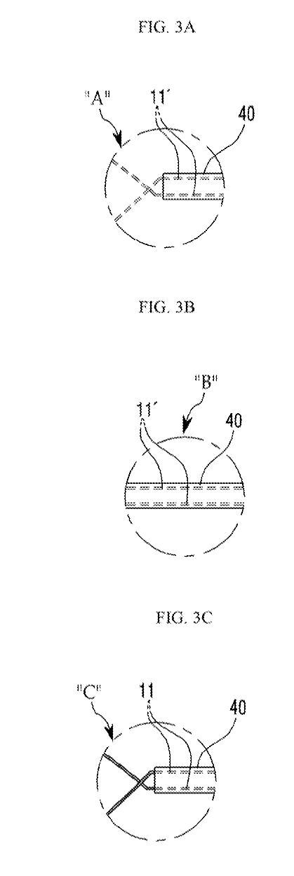

Further, as shown in FIGS. 3A to 3C, the power connection lines 11 and 11′ of the first and second separated bodies 10-1 and 10-2 may be formed of a ring (two lines), not a single line, to allow for steady power supply even when the power connection lines 11 and 11′ are partially shorted.

As shown in FIG. 4, the power connection lines 11, 11′, and 12 of the first, second, and third separated bodies 10-1, 10-2, and 10-3 may be formed of a ring (two lines), not a single line, and as shown in FIG. 5, the power connection lines 11, 11′, 12, and 12′ of the first, second, third, and fourth separated bodies 10-1, 10-2, 10-3, and 10-4 may be formed of a ring (two lines), not a single line.

The insulator 20 may be tubular and may be formed of a polytetrafluoroethylene (PTFE) material.

Further, the power connection lines of the separated bodies are extended to the outside of the separated bodies, and may thus be configured to be inserted and guided in an insulation 1 to polymer tube 40.

In this case, the insulation polymer may include any one of polyimide, Teflon™, and Nylon™ that are insulation material.

A power line 31 of the electricity generator 30 may be directly connected to the power connection lines 11 and 11′ of the first and second separated bodies 10-1 and 10-2, or after inserting the stent, the power line 31 may be indirectly connected to the power connection lines 11 and 11′ that are held by a separate assistant tool (clamp-type) that may be inserted into the lumen of the body.

Further, the first, second, third, and fourth separated bodies 10-1, 10-2, 10-3, and 10-4 may be formed by weaving superelastic shape memory alloy wires 2 in various manners to be extended or contracted in inner and outer directions and in a longitudinal direction. Various changes may be made to the shape, number, and length of the separated bodies and the connection structure of the power connection lines, and it should be appreciated that such changes in the structure belong to the scope of the present invention.

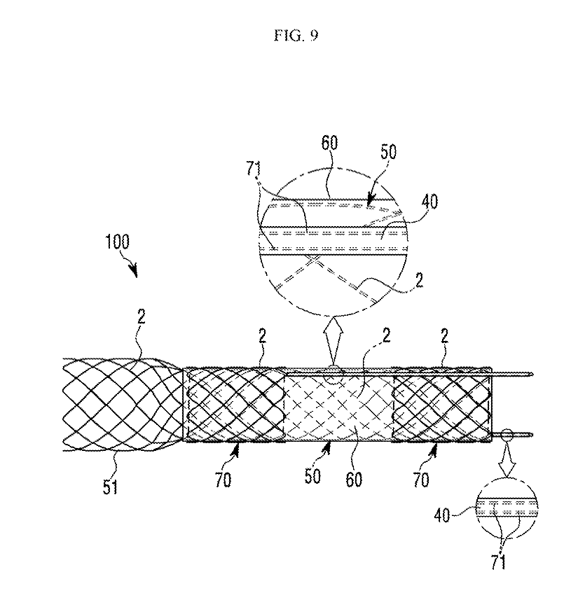

According to a second embodiment of the present invention, a stent 100 for electrothermal treatment, as shown in FIG. 9, includes a first hollow cylindrical body 50 that is formed by weaving or crossing superelastic shape memory alloy wires 2 in a mesh pattern and that is extended in a longitudinal direction. The first cylindrical body 50 further includes an expanded tube part 51 larger in diameter than the first cylindrical body 50 at an end thereof, which may be stuck in the lumen of the body.

The first cylindrical body 50 except for the expanded tube part 51 may be coated with an insulation material to form an insulation layer 60. Second hollow cylindrical bodies 70 smaller in length than the first cylindrical body 50 are formed at both sides, respectively, of the outer circumferential surface of the insulation layer 60, and the second cylindrical bodies 70 each are connected and fixed to the first cylindrical body 50.

For example, both sides of each second cylindrical body 70, where an inlet and an outlet are formed, are connected and fixed to the first cylindrical body 50 with the outer surface coated with the insulation layer 60 by treading in a circumferential direction of the first cylindrical body 50.

Alternatively, the outer circumferential surface of each second cylindrical body 70 may be connected and fixed to the first cylindrical body 50 with the outer surface coated with the insulation layer 60 by threading in a longitudinal direction, diagonal direction, or spiral direction.

In this case, the insulation layer 60 may include any one of parylene or silicone with excellent insulation.

The wires 2 are outwardly extended from first ends of the pair of the second cylindrical bodies 70 in an opposite direction of the expanded tube part 51 to form power connection lines 71 that are respectively connected to the power lines 31 of the electricity generator 30.

In this case, the power connection line 71 of the second cylindrical body 70 positioned closest to the expanded tube part 51 is formed to depart from the second cylindrical body 70 positioned farthest from the expanded tube part 51.

This is for the purpose of facilitating connection between the power lines 31 of the electricity generator 30 and the pair of the second cylindrical bodies 70.

In this case, the power connection lines 71 of the second cylindrical bodies 70 are configured to be inserted and guided in a tube 40 that is formed of insulation polymer and that may be inserted into the body while preventing contact with the skin, and ends of the power connection lines 71 are formed to be exposed.

This is for the purpose of supplying each electric current from the 30 to a respective one of the second cylindrical bodies 70 without overlap.

In this case, the insulation polymer may include any one of polyimide, Teflon™, and Nylon™ that are insulation material.

Each power connection line 71 is formed of a ring (two lines), not a single line.

The power connection lines 71 may be connected to the power lines 31 of the electricity generator 30 directly or indirectly by way of an assistant tool (clamp-type) that may be inserted into the lumen of the body and receive electric current.

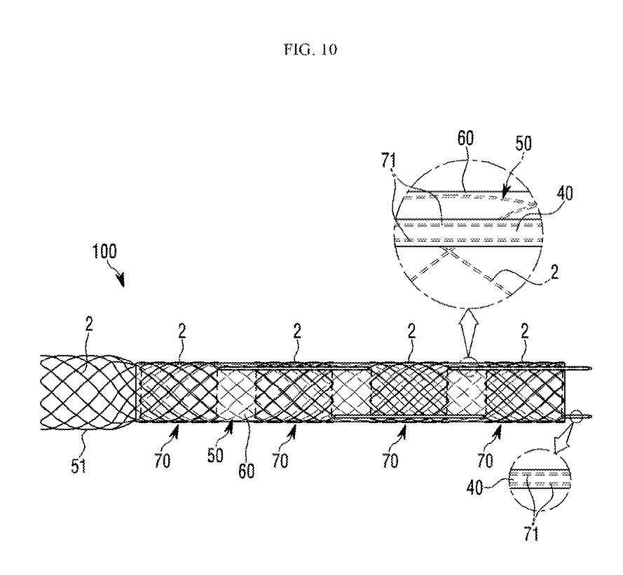

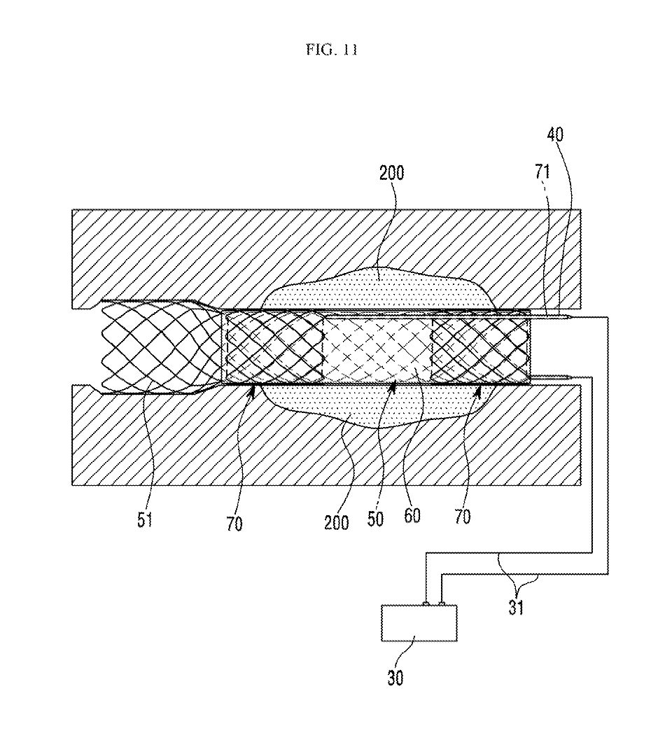

According to another second embodiment of the present invention, a stent 100 for electrothermal treatment, as shown in FIG. 10, includes a plurality of second cylindrical bodies 70 spaced apart from each other at a predetermined distance in a longitudinal direction of a first cylindrical body 50.

In this case, the number of second cylindrical bodies 70 may be configured to be larger than the number of second cylindrical bodies 70 shown in FIG. 9 as the length of the first cylindrical body 50 increases.

Among the plurality of second cylindrical bodies 70, odd-numbered ones 70 are connected with each other through power connection lines 71, and the power connection line 71 of the second cylindrical bodies 70 positioned last is connected to a power line 31 of an electricity generator 30 while extended outwards in an opposite direction of an expanded tube part 51.

Further, among the plurality of second cylindrical bodies 70, even-numbered ones 70, i.e., the remainder of the plurality of second cylindrical bodies 70 except for the odd-numbered second cylindrical bodies 70, are connected with each other through power connection lines 71, and the power connection line 71 of the second cylindrical body 70 positioned last is connected to another power line 31 of the electricity generator 30 while extended outwards in the opposite direction of the expanded tube part 51.

In this case, as described above in connection with FIG. 9, the insulation layer 60 may be formed of any one of parylene or silicone with excellent insulation. The power connection lines 71 of the second cylindrical bodies 70 are configured to be inserted and guided in tubes 40 formed of insulation polymer, which may be inserted into the body while preventing a contact with the skin.

The insulation polymer may include any one of polyimide. Teflon™, and Nylon™ that are insulation material.

As described above in connection with FIG. 9, each power connection line 71 may be formed of a ring (two lines), not a single line, and the power connection line 71 may be connected to the power line 31 of the electricity generator 30 directly or indirectly via an assistant tool (clamp-type) that may be inserted into the lumen of the body and that may receive electric current.

Although connections are made between second cylindrical bodies 70 that are not adjacent to each other, for example, such connections may be made between adjacent second cylindrical bodies 70 to generate electric heat according to the circumstance of the lesion portion 200.

The operations and actions of the present invention configured as described above are now described.

First, a stent 100 is inserted by a stent inserting device into a lesion portion 200, which is stenosed or obstructed by cancer tissue or malignant tumor in a lumen of the body, and a procedure is performed.

The stent 100 includes at least two or more separated bodies formed by separating in a longitudinal direction a hollow cylindrical body formed by weaving superelastic shape memory alloy wires 2 and an insulator 20 that is formed of a flexible material, such as, e.g., polytetrafluoroethylene (PTFE), and that connects the separated bodies with each other. The separated bodies are inserted into the stenosed or obstructed lesion portion 200 to expand the lesion portion 200, thus securing a passage.

Under such circumstance, an electricity generator 30 is connected to power connection lines that are formed by extending the wires 2 from the rear sides of the respective separated bodies, and thus, electric current flows between the separated bodies to generate electric heat. The lesion tissue may be cauterized by the electric heat.

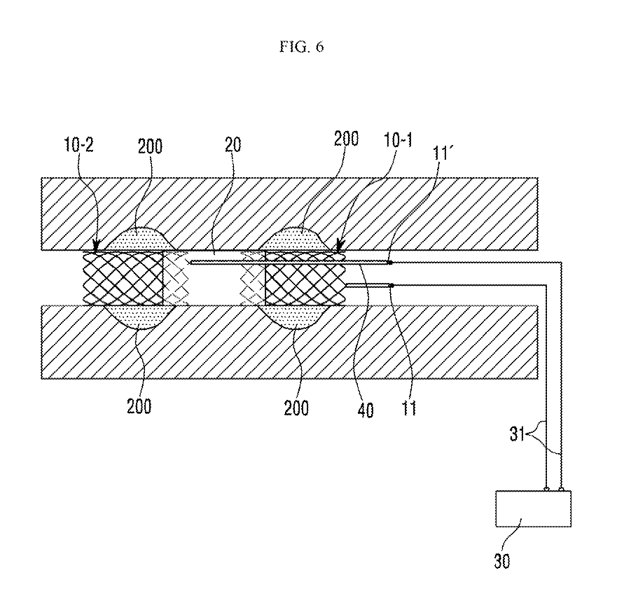

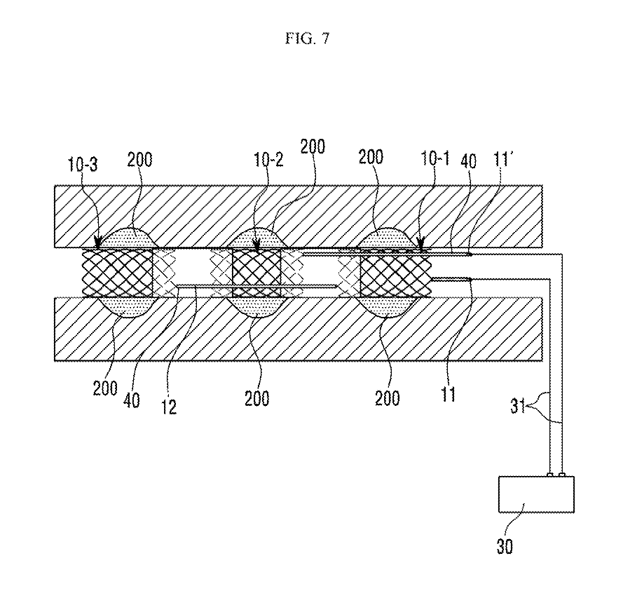

Specifically, in case the hollow cylindrical body is separated into two separated bodies, i.e., first and second separated bodies 10-1 and 10-2, if power lines 31 of the electricity generator are connected to the power connection lines 11 and 11′ formed by extending the wires 2 from the rear sides of the first and second separated bodies 10-1 and 10-2 as shown in FIG. 6, electric current flows between the first and second separated bodies 10-1 and 10-2 to generate electric heat, and the electric heat may cauterize the lesion tissue.

Further, in case the hollow cylindrical body is separated into three separated bodies, i.e., first, second, and third separated bodies 10-1, 10-2, and 10-3, the first and third separated bodies 10-1 and 10-3 are connected with each other via the power connection line 12 as shown in FIG. 7, if the power lines 31 of the electricity generator 30 are connected to the power connection lines 11 and 11′ formed by extending the wires 2 from the rear sides of the first and second separated bodies 10-1 and 10-2, electric current flows between the first, second, and third separated bodies 10-1, 10-2, and 10-3 to generate electric heat by which the lesion tissue may be cauterized.

Further, in case the hollow cylindrical body is separated into four separated bodies, i.e., first, second, third, and fourth separated bodies 10-1, 10-2, 10-3, and 10-4, and the first and third separated bodies 10-1 and 10-3 are connected with each other via the power connection line 12 while the second and fourth separated bodies 10-2 and 10-4 are connected with each other via the power connection line 12′, if the power lines 31 of the electricity generator 30 are connected to the power connection lines 11 and 11′ formed by extending the wires 2 from the rear sides of the first and second separated bodies 10-1 and 10-2, electric current flows between the first, second, third, and fourth separated bodies 10-1, 10-2, 10-3, and 10-4 to generate electric heat by which the lesion tissue may be cauterized.

As such, according to the first embodiment of the present invention, the stent 100 may prevent other unnecessary portions than the lesion portion 200 from being cauterized using the electric heat generated by allowing electric current to flow between two or more separated bodies and may perform a treatment with the cauterization of the lesion tissue limited to a minimum range.

In other words, the stent 100 according to the present invention may prevent electric current from flowing to other internal organs or skin than the lesion portion 200.

Further, various changes in the length or number of the separated bodies may be made depending on the length and area of the lesion portion 200, so that various lesion portions 200 may be cauterized by a procedure using the stent 100 according to the present invention.

Further, according to the present invention, the power connection lines 11, 11′, 12, and 12′ of the first, second, third, and fourth separated bodies 10-1, 10-2, 10-3, and 10-4 each may be formed of a ring (two lines), not a single line, and thus, even when a partial short circuit occurs in use, the connection to power may be maintained.

The insulator 20 connecting the separated bodies with each other may be shaped as a tube. The insulator 20 may be formed of an artificial vessel material (e.g., polytetrafluoroethylene; PTFE) to prevent any side effects that may occur during a procedure and to facilitate performing a procedure on a curved lumen.

Further, the power connection lines 11, 11′, 12, and 12′ of the first, second, third, and fourth separated bodies 10-1, 10-2, 10-3, and 10-4 are adapted to be inserted and guided in the tubes 40 formed of insulation polymer to prevent the power connection lines 11, 11′, 12, and 12′ from contacting the skin, so that unnecessary cauterization of other body portions may be prevented.

Further, as shown in FIG. 6, the electric current supplied to the power connection line 11′ passing through the outer circumferential surface of the first separated bodies 10-1 may be prevented from flowing to other separated bodies than the second separated bodies 10-2.

Further, as shown in FIG. 7, the electric current supplied to the power connection lines 11′ and 12 passing through the outer circumferential surfaces of the first and second separated bodies 10-1 and 10-2 may be prevented from flowing to other separated bodies than the second separated bodies 10-2 and the first and third separated bodies 10-1 and 10-3.

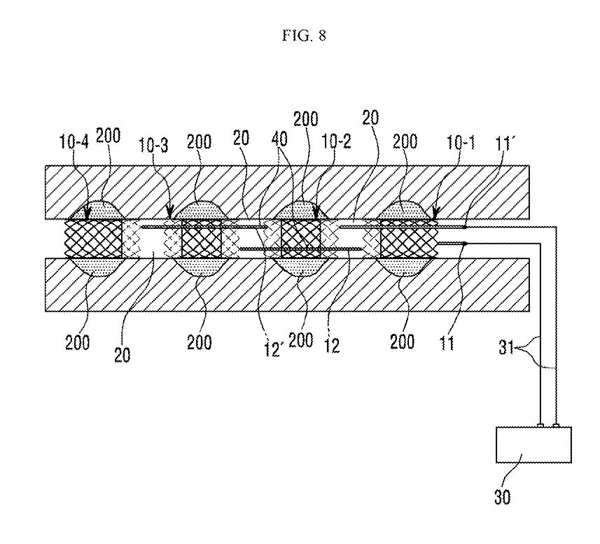

Further, as shown in FIG. 8, the electric current supplied to the power connection lines 11′, 12, and 12′ passing through the outer circumferential surfaces of the first, second, and third separated bodies 10-1, 10-2, and 10-3 may be prevented from flowing to other separated bodies than the first and third separated bodies 10-1 and 10-3 and the second and fourth separated bodies 10-2 and 10-4.

In other words, the power connection lines 11, 11′, 12, and 12′ of the first, second, third, and fourth separated bodies 10-1, 10-2, 10-3, and 10-4 are adapted to be inserted and guided in the tubes 40 formed of insulation polymer, thus preventing the electric current flowing across the power connection lines 11, 11′, 12, and 12′ from unnecessarily flowing to other separated bodies to generate electric heat.

For example, the insulation polymer may be formed of any one of polyimide, Teflon, and Nylon™ that are insulation material to prevent unnecessary current flow that may generate unnecessary electric heat.

The electricity generator 30 may be a high-frequency power supply, and the power lines 31 respectively connected with the power connection lines 11 and 11′ may be alternately changed between plus and minus in their polarities.

Meanwhile, as shown in FIG. 11, according to the second embodiment of the present invention, the stent 100 for electrothermal treatment is installed in the lumen of body which is positioned at the stenosed or obstructed lesion portion.

The expanded tube part 51 larger in diameter than the first cylindrical body 50 is stuck and fixed to the lumen of the body to expand the stenosed or obstructed lumen.

The expanded tube part 51 is stuck and fixed to the lumen of body without being slid on the stenosed or obstructed lesion portion by an external force that may occur outside or by body swinging.

The power lines 31 of the electricity generator 30 are connected to the power connection lines 71 of the second cylindrical bodies 70 so that electric current flows between a plurality of second cylindrical bodies 70 to generate electric heat by which the lesion tissue is cauterized.

In this case, since the power connection lines 71 are inserted in the tubes 40 formed of insulation polymer, unnecessary flow of electric current from the pair of second cylindrical bodies 70 may be prevented, so that electric heat may be unnecessarily generated.

For example, even when the power connection line 71 of the second cylindrical bodies 70 positioned closest to the expanded tube part 51 is positioned adjacent to other second cylindrical bodies 70, the insulation polymer tubes 40 may prevent the electric current supplied to the power lines 71 from flowing over to the other adjacent second cylindrical bodies 70.

In other words, the insulation polymer may include any one of polyimide. Teflon™, and Nylon™ that are insulation material to prevent unnecessary current flow and resultantly unnecessary generation of electric heat.

Further, the insulation layer 60 is formed of any one of parylene or silicone with excellent insulation, and may thus prevent an electric current from flowing from the second cylindrical bodies 70 receiving electric current from the electricity generator 30 to the first cylindrical body 50, thus preventing unnecessary generation of electric heat.

Further, each power connection line 71 is formed of a ring (two lines), not a single line, and thus, even when part of the power connection line 71 is shorted during the cauterization, supply of electric current to the second cylindrical bodies 70 may be continued.

Further, since the stent 100 for electrothermal treatment includes a first cylindrical body 50 and a pair of second cylindrical bodies 70, i.e., the stent 100 is configured “in double,” the stent 100 may cauterize the lesion tissue with the narrowed lumen in the body expanded more easily.

The electricity generator 30 may be a high-frequency power supply, and the power lines 31 respectively connected with the power connection lines 71 of the pair of second cylindrical bodies 70 may have positive polarity and negative polarity, respectively, or alternately, negative polarity and positive polarity, respectively.

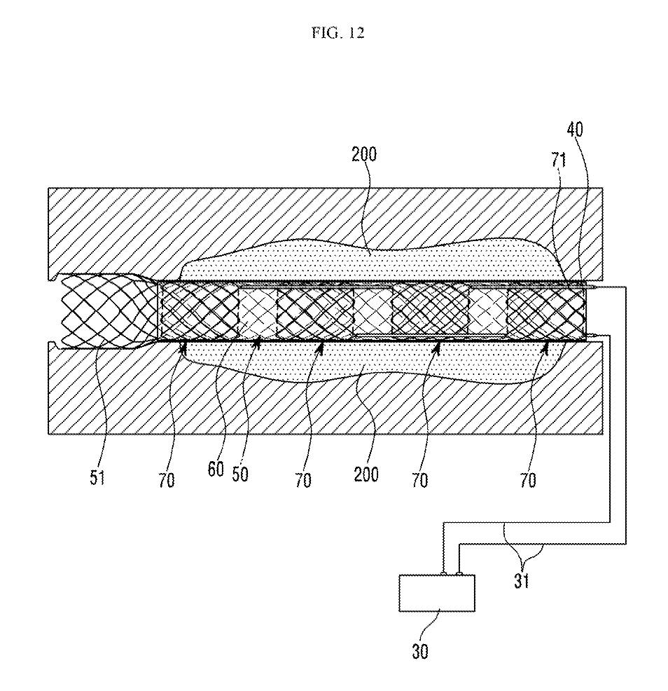

Meanwhile, in case, as shown in FIG. 12, the lesion portion is longer or broader than the stenosed or obstructed lesion portion shown in FIG. 11, the stent 100 for electrothermal treatment according to another second embodiment of the present invention is installed in the lumen of the body, which is positioned at the lesion portion.

For example, the narrowed lumen of the body may be expanded by a plurality of second cylindrical bodies 70 spaced apart from each other at a predetermined distance in a longitudinal direction of the first cylindrical body 50.

Among the plurality of second cylindrical bodies 70, the power connection line 71 of the odd-numbered second cylindrical body 70 positioned last is connected to a power line 31 of the electricity generator 30, and the power connection line 71 of the even-numbered second cylindrical bodies 70 positioned last is connected to another power line 31 of the electricity generator 30.

Then, electric current flows between the plurality of second cylindrical bodies 70 to generate electric heat by which the lesion tissue is cauterized.

For example, the cauterization using the electric heat may be performed on a longer or broader range of lesion tissue as compared with that is shown in FIG. 11.

In this case, the power connection lines 71 are inserted in the tubes 40 formed of insulation polymer, and thus, unnecessary flow of electric current between the odd-numbered second cylindrical bodies 70 and the even-numbered second cylindrical bodies 70 may be prevented, and thus unnecessary generation of electric heat may be prevented.

For example, electric current may be prevented from flowing from the power connection lines 71 connected with the odd-numbered second cylindrical bodies 70 to the even-numbered second cylindrical bodies 70 or from the power connection lines 71 connected with the even-numbered second cylindrical bodies 70 to the odd-numbered second cylindrical bodies 70.

In other words, the insulation polymer may be formed of any one of polyimide, Teflon™, and Nylon™ that are insulation material, and thus, unnecessary flow of electric current and unnecessary generation of electric heat may be prevented.

Further, since the insulation layer 60 is formed of any one of parylene or silicone with excellent insulation, electric current may be prevented from the plurality of second cylindrical bodies 70 receiving electric current from the electricity generator to the first cylindrical body 50, thus preventing electric heat from being unnecessarily generated.

Further, since each power connection line 71 is formed of a ring (two lines), not a single line, even when the power connection line 71 is partially shorted during the cauterization, supply of electric current to the plurality of second cylindrical bodies 70 may be continued.

Further, since the stent 100 for electrothermal treatment includes a first cylindrical body 50 and a plurality of second cylindrical bodies 70, i.e., configured “in double,” the cauterization may be performed on a broader or longer range of lesion tissue as compared with the lesion portion shown in FIG. 1, with the narrowed lumen of body expanded more easily.

The electricity generator 30 may be a high-frequency power supply, and the power lines 31 respectively connected with the power connection lines 71 of the second cylindrical bodies 70 positioned last at the odd-numbered and even-numbered locations may have polarities alternating between positive and negative.

Although the present invention has been shown and described in connection with exemplary embodiments thereof, it will be understood by one of ordinary skill in the art that various changes in form or detail may be made thereto without departing from the scope of the present invention defined in the following claims.

What is claimed is:

1. A stent for electrothermal treatment, the stent inserted into a stenosed or obstructed lesion portion that occurs in a lumen of a body to expand the lesion portion and applying electric heat to the entry point to cauterize a lesion portion, the stent comprising at least two or more separated bodies formed by separating in a longitudinal direction a hollow cylindrical body formed by weaving superelastic shape memory alloy wires, the separated bodies connected with each other through an insulator formed of a flexible material, wherein the separated bodies respectively include power connection lines formed by extending the wires from rear sides thereof, and wherein when an electricity generator is connected to the power connection lines of the separated bodies, an electric current flows between the separated bodies to generate electric heat by which the lesion tissue is cauterized.

2. The stent of claim 1, wherein the separated bodies include a first separated bodies 10-1 and a second separated bodies, wherein power connection lines are formed by extending the wires from rear sides of the first and second separated bodies, respectively, and wherein when the electricity generator is connected with the power connection lines of the first and second separated bodies, an electric current flows between the first and second separated bodies to generate electric heat by which the lesion tissue is cauterized.

3. The stent of claim 1, wherein the separated bodies include a first separated body, a second separated body, and a third separated body, wherein power connection lines are formed by extending the wires from rear sides of the first and second separated bodies, respectively, wherein a power connection line formed by extending the wire from a rear side of the third separated body is connected to a front side of the first separated body, and wherein when the electricity generator is connected to the power connection lines of the first and second separated bodies, an electric current flows between the first, second, and third separated bodies to generate electric heat by which the lesion tissue is cauterized.

4. The stent of claim 1, wherein the separated bodies include a first separated body, a second separated body, a third separated body, and a fourth separated body, wherein power connection lines are formed by extending the wires from rear sides of the first and second separated bodies, respectively, wherein a power connection line formed by extending the wire from a rear side of the third separated body is connected to a front side of the first separated body, and a power connection line formed by extending the wire from a rear side of the fourth separated body is connected to a front side of the second separated body, and wherein when the electricity generator is connected with the power connection lines of the first and second separated bodies, an electric current flows between the first, second, third, and fourth separated bodies to generate electric heat by which the lesion portion is cauterized.

5. The stent of claim 1, wherein the power connection lines each are formed in a ring shape.

6. The stent of claim 1, wherein the power connection lines of the separated bodies each are configured to be inserted and guided in a tube that may be inserted into the body while preventing a contact to skin and that is formed of an insulation polymer.

7. The stent of claim 6, wherein the insulation polymer is formed of any one of polyimide, Teflon™, and Nylon™.

8. The stent of claim 1, wherein the insulator is formed of an artificial vessel material (polytetrafluoroethylene; PTFE) in a tube shape.

9. A stent for electrothermal treatment, the stent comprising a first hollow cylindrical body formed long in a longitudinal direction by weaving or crossing superelastic shape memory alloy wires in a mesh pattern, an end of the first cylindrical body having an expanded tube part larger in diameter than the first cylindrical body to be stuck in a lumen of a body, wherein an insulation layer is formed by coating the first separated bodies with an insulation material, wherein second hollow cylindrical bodies shorter than the first cylindrical body are formed at both sides, respectively, of an outer circumferential surface of the insulation layer, the second cylindrical bodies connected and fixed to the first cylindrical body, wherein power connection lines formed by extending outwards the wires from first ends of the pair of second cylindrical bodies are connected with power lines, respectively, of an electricity generator, and wherein after placing the first cylindrical body on a lesion portion with the expanded tube part stuck in the lumen of the body to thereby expand the narrowed lumen of the body, the power lines of the electricity generator are connected to the power connection lines of the second cylindrical bodies to allow an electric current to flow between the pair of second cylindrical bodies to generate electric heat by which a lesion tissue is cauterized.

10. The stent of claim 9, wherein the second cylindrical bodies include a plurality of second cylindrical bodies spaced apart from each other at a predetermined distance in a longitudinal direction of the first cylindrical body, wherein among the plurality of second cylindrical bodies, odd-numbered second cylindrical bodies are connected with each other via power connection lines so that the power connection line of the second cylindrical body positioned last among the odd-numbered second cylindrical bodies is connected to a power line, and the other second cylindrical bodies are connected with each other via power connection lines so that the power connection line of the second cylindrical body positioned last among the other second cylindrical bodies is connected to another power line.

11. The stent of claim 9, wherein the insulation layer is formed of any one of parylene or silicone.

12. The stent of claim 9, wherein the power connection lines of the second cylindrical bodies each are configured to be inserted and guided in a tube that may be inserted into the body while preventing a contact to skin and that is formed of an insulation polymer.

13. The stent of claim 12, wherein the insulation polymer is formed of any one of polyimide, Teflon™, and Nylon™.

14. The stent of claim 9, wherein the power connection lines each are formed in a ring shape.
